(19) United States
(12) Patent Application Publication
(10) Pub. No.: US 2007/0142715 A1
(43) Pub. Date: Jun. 21, 2007

(54) CHEST STRAP FOR MEASURING VITAL SIGNS

(75) Inventors: Matthew John Banet, Del Mar, CA (US); Michael James Thompson, San Diego, CA (US); Zhou Zhou, La Jolla, CA (US)

Correspondence Address: Triage Wireless, Inc.;Matthew John Banet, 6540 LUSK BLVD., C200, SAN DIEGO, CA, 92121, US

(73) Assignee: Triage Wireless, Inc., San Diego, CA (US)

(21) Appl. No.: 11/306,243

(22) Filed: Dec. 20, 2005

(51) Int. Cl. A61B 5/00; A61B 5/02

(52) U.S. Cl. 600/301; 600/323; 600/500; 600/485; 128/903

(57) ABSTRACT

The invention provides a monitor featuring a chest strap that measures a variety of different vital signs (e.g., heart rate, blood pressure, and pulse oximetry) and wirelessly transmits them to an external device. The chest strap features: i) an electrode system with at least two electrodes that generate electrical signals to generate an ECG waveform; ii) an optical component featuring a light source and a photodetector that generate an optical waveform; iii) a processing component that receives and processes the ECG and optical waveforms to generate vital sign parameters, e.g. heart rate, pulse oximetry, and systolic and diastolic blood pressure; and iv) a wireless transmitter that receives the vital sign parameters from the processing component and wirelessly transmits them to the external device.

### BACKGROUND OF THE INVENTION

The present invention relates to a chest strap that measures vital signs, such as heart rate, pulse oximetry, and blood pressure, for medical and exercise applications.

### DESCRIPTION OF THE RELATED ART

Chest straps are used in several monitors to measure a user's heart rate and the heart's electrical activity during exercise. Typically such chest straps feature two or more rubber or cloth electrodes that detect electrical signals corresponding to each beat of the user's heart. An amplifier circuit, typically embedded within the chest strap and powered by a battery, receives the electrical signals and processes them to generate an ECG waveform similar to a conventional electrocardiogram (‘ECG’). A processor in electrical communication with the amplifier circuit processes the ECG waveform to determine a heart rate. Typically the chest strap additionally includes a short-range wireless transmitter that sends the heart rate to a body-worn component (e.g. a wrist watch) that includes a matched wireless receiver. The body-worn component displays the heart rate so that the user can monitor it during exercise.

Various methods have been disclosed for using ECG chest straps to obtain a heart rate. One such method is disclosed in Nissila et al., U.S. Pat. No. 6,775,566. The ′566 patent discloses a system and method for determining heart rate from a chest strap worn during exercise that features two electrodes. The chest strap relays the information to a wrist-worn device through an optical or wireless interface. Bimbaumn, U.S. Pat. No. 6,605,044, describes a heart rate monitor that includes a system and method to determine caloric expenditure during exercise through an array of inputs.

Rytky, U.S. Pat. No. 6,553,247, discloses a system and method for monitoring heart rate in sports and medicine. The electrode belt wraps around the patient's chest and transmits processed electrical signals to an external computer.

Sham et al., U.S. Pat. No. 5,891,042, discloses a fitness-monitoring device that includes a pedometer for measuring steps and a wireless heart rate monitor to determine exertion levels.

Asai et al., U.S. Pat. No. 4,681,118, discloses a waterproof electrode system with a transmitter for recording an electrocardiogram while the user is exercising in the water.

Jimenez et al., U.S. Pat. No. 4,367,752, discloses a monitoring system comprising a computer for determining heart rate through multiple reference points using electrodes. Along with heart rate, the system analyses fitness, calories consumed, and time elapsed.

### SUMMARY OF THE INVENTION

In one aspect, the invention provides a system featuring a chest strap and external monitor that measures a variety of different vital signs (e.g., heart rate, blood pressure, and pulse oximetry). The chest strap features: i) an electrode system with at least two electrodes that detect electrical signals to generate an ECG waveform; ii) an optical component featuring a light source and a photodetector that detect optical signals to generate an optical waveform; iii) a processing component that receives and processes the ECG and optical waveforms to generate vital sign parameters, e.g. heart rate, pulse oximetry, and systolic and diastolic blood pressure; and iv) a wireless transmitter that receives the vital sign parameters from the processing component and wirelessly transmits them to the external monitor, such as a body or wrist-worn monitor, or a laptop computer. In another aspect, the invention provides a system for measuring vital signs from a patient that features: i) a chest strap including at least two electrodes connected to an amplifier circuit and configured to generate an electrical signal; ii) an optical sensor, connected to or included within the chest strap, featuring at least one light source and a photodetector configured to generate an optical signal; iii) a processor in electrical communication with the amplifier circuit and the optical sensor and configured to receive the optical and electrical signals and process these signals with an algorithm to determine the patient's vital signs.

In embodiments, the optical sensor includes two light sources, e.g. a first light source that emits radiation in the red spectral region (e.g. λ=600-700 nm), and a second light source that emits radiation in the infrared spectral region (e.g. λ=800-1100 nm). The optical sensor is typically configured to generate a separate optical signal corresponding to each light source. To calculate pulse oximetry, the processor further comprises an algorithm for processing the separate optical signals corresponding to each light source. The processor further typically includes an algorithm for processing the electrical signals (or, alternatively, the optical signals) to calculate heart rate. For example, this algorithm may include a Fourier Transform algorithm or a peak-detecting algorithm that extract a heart rate from the electrical signals.

In another embodiment, the processor also includes an algorithm that processes the electrical signal in combination with the optical signal to calculate a blood pressure value. For example, in one embodiment, the processor includes an algorithm that determines blood pressure by processing: 1) a first time-dependent feature of the optical signal; 2) a second time-dependent feature of the electrical signal; and 3) a calibration parameter. As is described in more detail below, a time difference between features of the optical and electrical signals correlates to both systolic and diastolic blood pressure.

In other embodiments, the chest strap includes a short-range wireless transmitter to transmit the vital signs to an external monitor. For example, the external monitor can be a body-worn component, a watch component, or a laptop computer. In these cases, the external monitor includes a matched wireless receiver configured to receive the vital signs from the wireless transmitter.

The chest strap and external monitor are easily worn by the patient during periods of exercise or day-to-day activities, and make non-invasive measurements of vital signs in a matter of seconds. The resulting information has many uses for patients, medical professionals, insurance companies, pharmaceutical agencies conducting clinical trials, and organizations for home-health monitoring.

These and other advantages are described in detail in the following description, and in the claims.

### BRIEF DESCRIPTION OF THE DRAWINGS

FIG. 1 is a semi-schematic view of a chest strap and external monitor for measuring a patient's vital signs according to the invention;

FIG. 2 is a schematic view of an optical component featuring optical sensors that connects to, or is comprised by, the chest strap of FIG. 1;

FIG. 3 is a graph of time-dependent optical and ECG waveforms, generated by the chest strap and optical component of FIGS. 1 and 2, that are processed to calculate the patient's vital signs;

FIG. 4 is a semi-schematic view of the chest strap of FIG. 1 in wireless communication with both a weight scale and the external monitor; and

FIG. 5 is a schematic view of an Internet-based system coupled with the chest strap of FIG. 1 that transmits vital sign information through a wireless network to an Internet-accessible computer system.

### DETAILED DESCRIPTION OF THE INVENTION

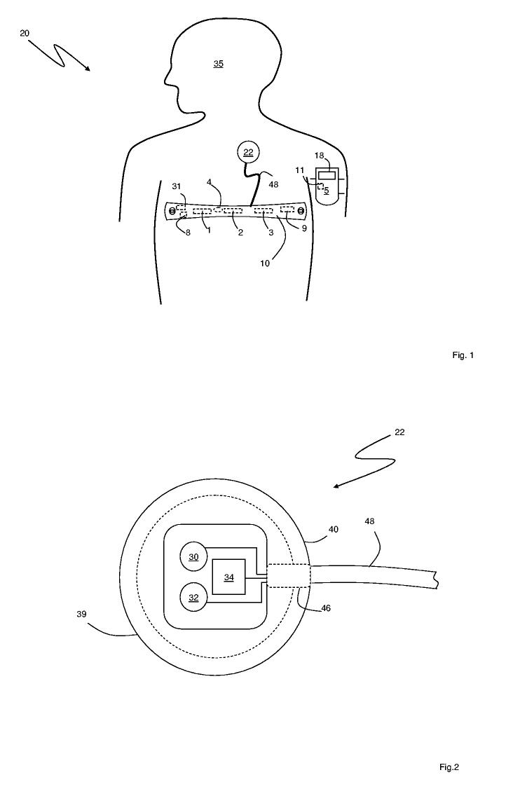

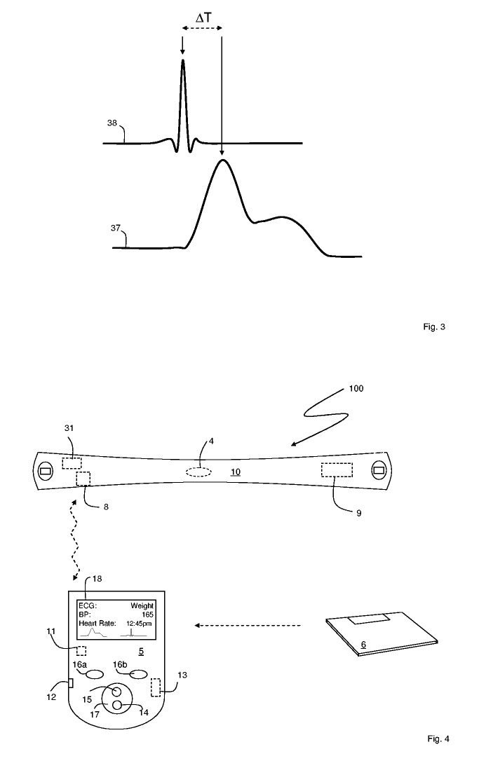

FIG. 1 shows a chest strap 10 according to the invention featuring an electrode system 1, 2, 3 that measures electrical signals corresponding to each heartbeat of the user 35. The electrode system 1, 2, 3, for example, typically features two signal electrodes and a ground electrode, each composed of a conductive rubber or fabric. The chest strap 10 includes an amplifier circuit 31 connected to the electrode system 1, 2, 3 that receives and processes the electrical signals and, in response, generates an analog ECG waveform. A processor 4 (e.g., a microprocessor) connected to the amplifier circuit 31 receives the ECG waveform and digitizes it to generate a computer-readable series of data points representing the ECG waveform. Referring also to FIG. 2, the chest strap 10 connects to an optical component 22 featuring separate light sources 30, 32 and a photodetector 34 that measures an optical waveform, also called a plethysmogram, from an underlying artery in the patient 35. The optical component 22 can also be embedded in the chest strap. A battery (not shown in the figure) powers the above-described systems.

Both the optical and ECG waveforms feature a ‘pulse’, described in detail below with respect to FIG. 3, corresponding to each heart beat. As with the ECG waveform, the processor 4 receives the optical waveform and digitizes it to generate a similar computer-readable series of data points representing the optical waveform. The processor 4 then processes the data points representing the optical and ECG waveforms, as described in detail below, to measure the user's vital signs, e.g. systolic and diastolic blood pressure, heart rate, and pulse oximetry values.

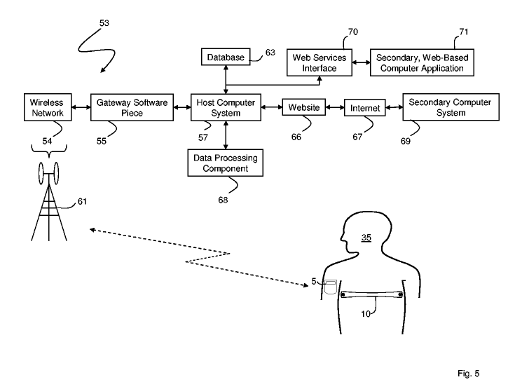

A wireless transceiver 8 embedded in the chest strap 10 receives the vital signs from the processor 4 and transmits them in the form of a packet to a matched wireless transceiver 11 within an external monitor 5, which is typically worn on the patient's body. For example, as shown in FIG. 4, the external monitor 5 may attach to the user's arm, and have a form factor similar to a conventional personal digital assistant (PDA) or pager. Alternatively the external monitor 5 may take the shape of a watch. The external monitor 5 includes an easy-to-read display 18 which renders the values of the vital signs so the user or a medical professional can easily read them. In addition, as described in more detail below with reference to FIG. 4, the wireless transceiver 11 within the external monitor 5 can wirelessly receive information from other devices, e.g. weight from a specially outfitted scale that includes a wireless transceiver. The external monitor 5 can also include a USB port 12, thereby allowing it to connect through a personal computer to an Internet-accessible website.

The optical component 22 that connects to or is embedded in the chest strap 10 features a pair of LEDs 30, 32 and photodetector 34 that, when attached to a patient, generate an optical waveform (37 in FIG. 3) using a ‘reflection mode’ optical configuration. The electrode system 1, 2, 3 in the chest strap 10 generates an ECG waveform (38 in FIG. 3). The optical waveform, once generated, passes through a cable 48 to the processor 4, which analyzes it in combination with the ECG waveform as described in detail below to measure a patient's systolic and diastolic blood pressure, heart rate, and pulse oximetry. The optical component 22 features an adhesive component 39 that adheres to the patient's skin and secures the LEDs 30, 32, and photodetector 34 in place to minimize the effects of motion. During operation, the cable 48 snaps into a plastic header 46 disposed on a top portion of the optical component 22. Both the cable 48 and header 46 include matched electrical leads that supply power and ground to the LEDs 30, 32, and photodetector 34.

To measure blood pressure, heart rate, and pulse oximetry, the LEDs 30, 32 generate, respectively, red and infrared radiation that irradiates an underlying artery. Blood volume increases and then decreases as the heart pumps blood through the patient's artery. Blood cells absorb and transmit varying amounts of the red and infrared radiation depending the on the blood volume and how much oxygen binds to the cells′ hemoglobin. The photodetector 34 detects a portion of the radiation that reflects off an underlying artery. In response an intermediary circuit converts that photocurrent to a stable voltage difference that is processed by an analog-to-digital converter embedded within the processing module. The analog-to-digital converter digitizes the photocurrent to generate a time-dependent optical waveform for each wavelength. In addition, the processor 4 analyzes waveforms generated at both red and infrared wavelengths, and compares a ratio of the relative absorption to a calibration table coded in its firmware to determine pulse oximetry according to processes known in the art. The processor additionally analyzes the time-dependent properties of one of the optical waveforms to determine the patient's heart rate.

Concurrent with measurement of the optical waveform, the electrode system detects an electrical impulse from the patient's skin that the processor processes to generate an ECG waveform. The electrical impulse is generated each time the patient's heart beats. Analysis of the optical and ECG waveforms is described in more detail in U.S. patent application Ser. No. 10/906,314, filed Feb. 14, 2005 and entitled PATCH SENSOR FOR MEASURING BLOOD PRESSURE WITHOUT A CUFF, the contents of which are incorporated herein by reference.

FIG. 3 shows both optical 37 and ECG 38 waveforms generated by the chest strap and optical component of FIGS. 1 and 2. Following a heartbeat, the electrical impulse travels essentially instantaneously from the patient's heart, where the electrode system in the chest strap detects it to generate the ECG waveform 38. At a later time, a pressure wave induced by the same heartbeat propagates through the patient's arteries and arrives at the optical component, where the LEDs and photodetector detect it as described above to generate the optical waveform 37. The propagation time of the electrical impulse is independent of blood pressure, whereas the propagation time of the pressure wave depends strongly on pressure, as well as mechanical properties of the patient's arteries (e.g., arterial size, stiffness). The processor runs an algorithm that analyzes the time difference ΔT between the arrivals of these signals, i.e. the relative occurrence of a well-defined feature (e.g., a peak) the optical 37 and ECG 38 waveforms. Calibrating the measurement (e.g., with a conventional blood pressure cuff) accounts for patient-to-patient variations in arterial properties, and correlates ΔT to both systolic and diastolic blood pressure. This results in a calibration table. During an actual measurement, the calibration source is removed, and the processor analyzes ΔT along with other properties of the optical and ECG waveforms and the calibration table to calculate the patient's real-time blood pressure.

The processor can analyze other properties of the optical waveform 31 to augment the above-mentioned measurement of blood pressure. For example, the waveform can be ‘fit’ using a mathematical function that accurately describes the waveform's features, and an algorithm (e.g., the Marquardt-Levenberg algorithm) that iteratively varies the parameters of the function until it best matches the time-dependent features of the waveform. In this way, blood pressure-dependent properties of the waveform, such as its width, rise time, fall time, and area, can be calibrated as described above. After the calibration source is removed, the optical component and chest strap measure these properties along with ΔT to determine the patient's blood pressure.

Methods for processing optical and ECG waveforms to determine blood pressure without using a cuff are described in the following co-pending patent applications, the entire contents of which are incorporated by reference: 1) CUFFLESS BLOOD-PRESSURE MONITOR AND ACCOMPANYING WIRELESS, INTERNET-BASED SYSTEM (U.S.S.N 10/709,015; filed Apr. 7, 2004); 2) CUFFLESS SYSTEM FOR MEASURING BLOOD PRESSURE (U.S.S.N. 10/709,014; filed Apr. 7, 2004); 3) CUFFLESS BLOOD PRESSURE MONITOR AND ACCOMPANYING WEB SERVICES INTERFACE (U.S.S.N. 10/810,237; filed Mar. 26, 2004); 4) VITAL SIGN MONITOR FOR ATHLETIC APPLICATIONS (U.S.S.N; filed Sep. 13, 2004); 5) CUFFLESS BLOOD PRESSURE MONITOR AND ACCOMPANYING WIRELESS MOBILE DEVICE (U.S.S.N. 10/967,511; filed Oct. 18, 2004); and 6) BLOOD PRESSURE MONITORING DEVICE FEATURING A CALIBRATION-BASED ANALYSIS (U.S.S.N. 10/967,610; filed October 18, 2004); 7) PERSONAL COMPUTER-BASED VITAL SIGN MONITOR (U.S.S.N. 10/906,342; filed Feb. 15, 2005); 8) PATCH SENSOR FOR MEASURING BLOOD PRESSURE WITHOUT A CUFF (U.S.S.N. 10/906,315; filed Feb. 14, 2005); 9) SMALL-SCALE, VITAL-SIGNS MONITORING DEVICE, SYSTEM AND METHOD (U.S.S.N. 10/907,440; filed Mar. 31, 2005); 10) PATCH SENSOR SYSTEM FOR MEASURING VITAL SIGNS (U.S.S.N. 11/160957; filed Jul. 18, 2005); 11) WIRELESS, INTERNET-BASED SYSTEM FOR MEASURING VITAL SIGNS FROM A PLURALITY OF PATIENTS IN A HOSPITAL OR MEDICAL CLINIC (U.S.S.N. 11/162719; filed Sep. 20, 2005); and 12) HAND-HELD MONITOR FOR MEASURING VITAL SIGNS (U.S.S.N. 11/162742; filed Sep. 21, 2005).

Referring to FIG. 4, in embodiments the external monitor 5 includes an integrated pedometer circuit 13 that measures steps and, using an algorithm, calories burned. The pedometer circuit 13, for example, can include an accelerometer or ‘tilt switch’ to measure the user's steps or activity level. To receive information from external devices, the external monitor 5 also includes: i) a Universal Serial Bus (USB) connector 12 that connects and downloads information from other external devices with serial interfaces; and ii) a short-range wireless transceiver 13 that receives information such as body weight and percentage of body fat from an external scale 6. The patient views information from a liquid crystal display (LCD) display 18, and can interact with the external monitor 5 (e.g., reset or reprogram it) using a series of buttons 16a and 16b.

FIG. 5 shows a preferred embodiment of an Internet-based system 53 that operates in concert with the chest strap 10 and external monitor 5 to send information from a patient 35 through a wireless network 54 to a web site 66 hosted on an Internet-based host computer system 57. In this case the external monitor includes a wireless transmitter that operates on a nation-wide wireless network (e.g., Sprint). A secondary computer system 69 accesses the website 66 through the Internet 67. The system 52 functions in a bidirectional manner, i.e. the external monitor 5 can both send and receive data. Most data flows from the external monitor 5; using the same network, however, the monitor can also receive data (e.g., ‘requests’ to measure data or text messages) and software upgrades.

A wireless gateway 55 connects to the wireless network 54 and receives data from one or more mobile devices. The wireless gateway 55 additionally connects to a host computer system 57 that includes a database 63 and a data-processing component 68 for, respectively, storing and analyzing the data. The host computer system 57, for example, may include multiple computers, software pieces, and other signal-processing and switching equipment, such as routers and digital signal processors. The wireless gateway 55 preferably connects to the wireless network 54 using a TCP/IP-based connection, or with a dedicated, digital leased line (e.g., a frame-relay circuit or a digital line running an X.25 or other protocols). The host computer system 57 also hosts the web site 66 using conventional computer hardware (e.g. computer servers for both a database and the web site) and software (e.g., web server and database software).

During typical operation, the patient typically wears the external monitor 5 and chest strap 10 during exercise, or for a short period (e.g., 24 hours). For long-term monitoring (e.g. several months), the patient may wear the external monitor 5 and chest strap 10 for shorter periods of time during the day. To view information sent from the external monitor 5, the patient 35 or medical professional accesses a user interface hosted on the web site 66 through the Internet 67 from the secondary computer system 69. The system 53 may also include a call center, typically staffed with medical professionals such as doctors, nurses, or nurse practioners, whom access a care-provider interface hosted on the same website 66.

In an alternate embodiment, the host computer system 57 includes a web services interface 70 that sends information using an XML-based web services link to a secondary, web-based computer application 71. This application 71, for example, could be a data-management system operating at a hospital. The external monitor described above can be used to determine the patient's location using embedded position-location technology (e.g., GPS or network-assisted GPS within the wireless transmitter). In situations requiring immediate medical assistance, the patient's location, along with relevant medical data collected by the blood pressure monitoring system, can be relayed to emergency response personnel.

In other embodiments, the optical component may include a green LED (operating at wavelengths between 520 nm and 570 nm) to improve stability of the optical measurement made in reflection mode. Using this wavelength, the optical component can be connected to virtually any part of the patient's body, or alternatively can be embedded within the chest strap. The above-described system can be used for both medical applications (e.g., 24-hour heart rate and blood pressure monitoring) and athletic applications (e.g., characterizing an athlete's heart rate during an athletic activity).

The chest strap 10 can include an accelerometer that measures acceleration (e.g. steps) that can indicate physical activity, and thus an optical time to make a measurement. The accelerometer can also be used for artifact rejection or noise cancellation to improve the quality of data used for the above-described heart rate and blood pressure algorithms. Also, the envelope of an ECG waveform can be processed to determine respiration rate in the patient. In this case, a low-frequency modulation of the envelope indicates a respiration frequency. In still other embodiments, only two signal electrodes are used (i.e. there is no ground electrode) to determine an ECG waveform. In this case, a ‘notch’ filter may be used to remove noise normally reduced by the ground electrode.

Still other embodiments are within the scope of the following claims.

What is claimed is:

1. A system for measuring vital signs from a patient comprising:
a chest strap comprising a plurality of electrodes connected to an amplifier circuit and configured to generate an electrical signal;
an optical sensor, connected to the chest strap, comprising at least one light source and a photodetector configured to generate an optical signal;
a processor in electrical communication with both the amplifier circuit and the optical sensor and configured to receive the optical and electrical signals and process these signals with an algorithm to determine the patient's vital signs.

2. The system of claim 1, wherein the optical sensor comprises two light sources.

3. The system of claim 2, wherein a first light source comprises a component that emits radiation in the red spectral region, and the second light source comprises a component that emits radiation in the infrared spectral region.

4. The system of claim 2, wherein the optical sensor is configured to generate a separate optical signal corresponding to each light source.

5. The system of claim 4, wherein the processor further comprises an algorithm for processing the separate optical signals to calculate pulse oximetry.

6. The system of claim 1, wherein the processor further comprises an algorithm for processing the electrical signals to calculate heart rate.

7. The system of claim 1, wherein the processor further comprises an algorithm that processes the electrical signal and the optical signal to calculate a blood pressure value.

8. The system of claim 7, wherein the processor further comprises an algorithm that calculates blood pressure by processing: 1) a first time-dependent feature of the optical signal; 2) a second time-dependent feature of the electrical signal; and 3) a calibration parameter.

9. The system of claim 1, further comprising a short-range wireless transmitter configured to transmit the vital signs to an external monitor.

10. The system of claim 9, further comprising an external monitor comprising a wireless receiver configured to receive the vital signs from the wireless transmitter.

11. The system of claim 10, wherein the external monitor is a body-worn monitor.

12. The system of claim 10, wherein the external monitor is a laptop computer.

13. A system for measuring vital signs from a patient comprising:
a chest strap comprising a plurality of electrodes connected to an amplifier circuit and configured to generate an electrical signal;
an optical sensor, comprised by the chest strap, comprising at least one light source and a photodetector configured to generate an optical signal;
a processor in electrical communication with the amplifier circuit and the optical sensor and configured to receive the optical and electrical signals and process these signals with an algorithm to determine the patient's vital signs.

14. The system of claim 13, wherein the optical sensor comprises two light sources.

15. The system of claim 14, wherein a first light source comprises a component that emits radiation in the red spectral region, and the second light source comprises a component that emits radiation in the infrared spectral region.

16. The system of claim 14, wherein the optical sensor is configured to generate a separate optical signal corresponding to each light source.

17. The system of claim 16, wherein the processor further comprises an algorithm for processing the separate optical signals to calculate pulse oximetry.

18. The system of claim 1, wherein the processor further comprises an algorithm for processing the electrical signals to calculate heart rate.

19. The system of claim 13, wherein the processor further comprises an algorithm that processes the electrical signal and the optical signal to calculate a blood pressure value.

20. The system of claim 19, wherein the processor further comprises an algorithm that calculates blood pressure by processing: 1) a first time-dependent feature of the optical signal; 2) a second time-dependent feature of the electrical signal; and 3) a calibration parameter.

21. A system for measuring blood pressure from a patient comprising:
a chest strap comprising a plurality of electrodes connected to an amplifier circuit and configured to generate an electrical signal;
an optical sensor, comprised by the chest strap, comprising at least one light source and a photodetector configured to generate an optical signal;
a processor in electrical communication with the amplifier circuit and the optical sensor and configured to receive the optical and electrical signals and process these signals with an algorithm that determines blood pressure by processing: 1) a first time-dependent feature of the optical signal; 2) a second time-dependent feature of the electrical signal; and 3) a calibration parameter.
